(19) United States
(12) Patent Application Publication
(10) Pub. No.: US 2022/0386895 A1
(43) Pub. Date: Dec. 8, 2022

(54) METHOD AND APPARATUS FOR MEASURING THE PROSTATIC URETHRAL LENGTH

(71) Applicant: Prodeon Medical Corporation, Taipei (TW)

(72) Inventors: Yue-Teh Jang, Los Altos, CA (US); Yu-Shih Weng, Taipei (TW); Chun-Chia Juan, Taipei (TW); De-Yu Chu, Taipei (TW)

(73) Assignee: Prodeon Medical Corporation, Taipei (TW)

(21) Appl. No.: 17/831,675

(22) Filed: Jun. 3, 2022

Related U.S. Application Data
(60) Provisional application No. 63/196,371, filed on Jun. 3, 2021.

(51) Int. Cl. A61B 5/107; A61B 1/307

(52) U.S. Cl. CPC A61B 5/1076; A61B 5/1079; A61B 1/307

(57) ABSTRACT

Devices and methods are disclosed for determining prostatic urethral length. A measuring device having an elongated shaft member with associated markings may be advanced through a working channel of the cystoscope so that a positioning aid disposed on the elongated shaft member is located at a bladder neck of the patient. A first position of the elongated shaft member is determined using the markings. The elongated shaft member is then withdrawn to a second position at which the positioning aid is located at the patient's verumontanum. The prostatic urethral length is determined based at least in part on relative translational movement of the elongated shaft member between the first position and the second position using the markings.

### RELATED APPLICATIONS

This application claims priority to U.S. Provisional Application No. 63/196,371, filed Jun. 3, 2021. The priority of this application is expressly claimed, and the disclosure is hereby incorporated by reference in its entirety.

### FIELD OF THE PRESENT DISCLOSURE

This disclosure relates to devices for managing or treating body tissues obstructing a hollow body lumen, such as the prostatic lobe tissues obstructing the urethra.

### BACKGROUND

The prostate is a walnut-shaped gland that wraps around the urethra through which urine is expelled from the bladder and plays a crucial role in the reproductive system of men. Although the gland starts out small, it tends to enlarge as a man ages. An excessively enlarged prostate results in a disease known as benign prostatic hyperplasia (BPH). Benign prostatic hyperplasia (BPH) refers to the abnormal, but non-malignant (non-cancerous) growth of the prostate observed very commonly in aging men. BPH is a chronic condition and is associated with the development of urinary outflow obstruction or luminal narrowing in the prostatic urethra. Bladder outlet obstruction (BOO) refers to a blockage at the base of the bladder that reduces or stops the flow of urine into the urethra and may be secondary to BPH. A range of related disorders referred to collectively as Lower Urinary Tract Symptoms (LUTS) can result, including sexual dysfunction, frequent urination, difficulty in voiding urine, urinary retention, urinary leakage, and urinary tract and bladder infections that worsen as the abnormal growth in the prostate enlarges and progresses.

It is estimated that approximately one in five adults report moderate-to-severe LUTS. These urinary storage and voiding problems substantially decrease the quality of life and is associated with various health conditions. Presently, there are a variety of medical devices, such as implants or stents, to aid in the control of urinary outflow obstruction. In order to achieve therapeutic effect with less pain in patient and optimize patient's quality of life, such implants or stents must be accurately sized according to the length of the patient's prostatic urethra. In addition to the suitable size of medical device chosen for intended patients, the length of patient's prostatic urethra is also served as predictive factor for surgical treatment of BPH. In some diagnosis procedure, the length of prostatic urethra is also a key item for check to anticipate the association with the degree of symptoms.

Accordingly, it would be desirable to provide a medical device and method for measuring the length of the patient's urethra with features of ease of use, satisfactory accuracy of measurement, being comfortable to patient.

### SUMMARY

This disclosure is directed to a measuring device for determining prostatic urethral length. The device may include an elongated shaft member configured to be introduced through a working channel of a cystoscope, a positioning aid disposed on the elongated shaft member and markings associated with the elongated shaft configured to indicate relative translational movement within the cystoscope.

In one aspect, the positioning aid may be at least one of a visual cue and a tactile cue.

In one aspect, the positioning aid may be deployable from a side of the elongated shaft member. The positioning aid may deploy automatically when unconstrained.

In one aspect, the positioning aid may be an expander that transitions between collapsed and expanded configurations. An indicator positioned proximal of the expander may be configured to correspond to a bladder neck position when the expander is drawn into contact with a patient's bladder.

In one aspect, the measuring device may have an emitter positioned at a distal end of the elongated shaft member configured to guide light to the distal end.

In one aspect, the markings may be on a distal portion of the elongated shaft member.

In one aspect, the markings may be on a transparent barrel through which the elongated shaft member is disposed. The barrel may be configured to be secured to the cystoscope.

This disclosure also includes a method for determining prostatic urethral length. The method may involve positioning a cystoscope so that a distal end of the cystoscope is at a location corresponding to a patient's verumontanum, advancing an elongated shaft member from within a working channel of the cystoscope, locating a positioning aid disposed on the elongated shaft member at a bladder neck of the patient such that the elongated shaft member is at a first translational position, corresponding the first position of the elongated shaft member to markings associated with the elongated shaft member, withdrawing the elongated shaft member until the positioning aid is located at the patient's verumontanum such that the elongated shaft member is at a second translational position, corresponding the second position of the elongated shaft member to markings associated with the elongated shaft member and determining prostatic urethral length based at least in part on relative translational movement of the elongated shaft member between the first position and the second position.

In one aspect, the positioning aid may be deployed from a side of the elongated shaft member.

In one aspect, at least one of locating the positioning aid at the bladder neck and withdrawing the elongated shaft until the positioning aid is located at the patient's verumontanum may be performed under visualization through the cystoscope. As such, the method may also involve guiding light to an emitter at a distal end of the elongated shaft member to aid the visualization.

In one aspect, locating the positioning aid at the bladder neck may be based at least in part on a tactile cue. As such, the method may involve expanding an expander at a distal end of the elongated shaft member within a patient's bladder to locate the positioning aid at the bladder neck. The expander may be collapsed prior to withdrawing the elongated shaft member, such that withdrawing the elongated shaft until the positioning aid is located at the patient's verumontanum comprises aligning an indicator proximal of the expander with the patient's verumontanum.

In one aspect, the elongated shaft member may be withdrawn through a transparent barrel connected to the cystoscope such that the markings are on the barrel.

This disclosure also includes a measuring device for determining prostatic urethral length, with a ruler portion having markings configured to indicate relative translational movement of a cystoscope and a stabilizer configured to position the ruler portion with respect to a patient.

In one aspect, the stabilizer may be configured to engage a penis. The stabilizer may be configured to engage a glans of the penis. The stabilizer may transition from an open configuration to a closed configuration when engaging the glans. The measuring device may also have a supporter configured to engage a more distal portion of the penis. The supporter may be a hinged collar.

In one aspect, the stabilizer may be a patch configured to be adhered to hypogastrium skin or may be configured as a belt.

In one aspect, the stabilizer may be an adjustable strip configured to encircle a base of the penis.

This disclosure also includes a method for determining prostatic urethral length. The method may involve positioning a ruler portion of a measuring device having markings configured to indicate relative translational movement of a cystoscope with respect to a patient using a stabilizer, locating a distal end of the cystoscope is at a patient's bladder neck such that the cystoscope is at a first translational position, corresponding the first position of the cystoscope with the markings, withdrawing the cystoscope until the distal end is located at the patient's verumontanum such that the cystoscope is at a second translational position, corresponding the second position of the cystoscope to the markings and determining prostatic urethral length based at least in part on relative translational movement of the cystoscope between the first position and the second position.

In one aspect, the stabilizer may be transitioned from an open configuration to a closed configuration to engage a glans of the penis. A supporter may be provided to engage a more distal portion of the penis. The supporter may be a hinged collar, such that the hinged collar is closed around the more distal portion of the penis.

In one aspect, the stabilizer may be adhered to hypogastrium skin to position the ruler portion.

In one aspect, the stabilizer may be belted around a torso of the patient to position the ruler portion.

In one aspect, the stabilizer may be an adjustable strip, such that a base of the penis is encircled to position the ruler portion.

In one aspect, a marker clip may be releasably attached to the cystoscope after locating the distal end of the cystoscope at the patient's bladder neck so that corresponding the first position and the second position to the markings is made in relation to the marker clip.

Still further, this disclosure also includes a measuring device for determining prostatic urethral length, with a ruler portion having markings configured to indicate relative translational movement of a cystoscope and a clip connected to the ruler portion configured to be removably attached to a cystoscope.

In one aspect, the ruler portion may be connected to the clip by a hinge. The hinge may be configured to allow the ruler portion to align longitudinally with the cystoscope when the clip is attached.

In one aspect, markings of the ruler portion may represent absolute measurements. Alternatively, markings of the ruler portion may represent relative measurements.

In one aspect, the clip may be configured to be attached to the cystoscope at a reference position relative to a patient.

This disclosure also includes a method for determining prostatic urethral length. The method may locating a distal end of the cystoscope at a patient's bladder neck such that the cystoscope is at a first translational position, releasably attaching a ruler portion of a measuring device having markings configured to indicate relative translational movement of a cystoscope with respect to a patient using a stabilizer, corresponding the first position of the cystoscope with the markings, withdrawing the cystoscope until the distal end is located at the patient's verumontanum such that the cystoscope is at a second translational position, corresponding the second position of the cystoscope to the markings and determining prostatic urethral length based at least in part on relative translational movement of the cystoscope between the first position and the second position.

In one aspect, the ruler portion may be releasably attached to a reference position relative to the patient when the distal end of the cystoscope is located at the patient's bladder neck. Accordingly, corresponding the second position of the cystoscope to the markings to determine prostatic urethral length may involve comparing the markings of the ruler portion with the reference position relative to the patient.

In one aspect, the ruler portion may be pivoted at a hinge of the clip to align the ruler portion longitudinally with the cystoscope when corresponding the second position of the cystoscope to the markings.

### BRIEF DESCRIPTION OF THE DRAWINGS

Further features and advantages will become apparent from the following and more particular description of the preferred embodiments of the disclosure, as illustrated in the accompanying drawings, and in which like referenced characters generally refer to the same parts or elements throughout the views, and in which:

FIG. 1 is a cross-section of the male anatomy comprising the lower portion of the bladder, showing the prostatic urethra length as extending from the bladder neck to the verumontanum.

FIG. 2 a schematic detail view of a distal end of a cystoscope with a measuring device disposed within a working channel according to an embodiment.

FIG. 3 a schematic view of a distal end of a measuring device deployed within a patient's urethra according to an embodiment.

FIGS. 4a-c are schematic views that demonstrate locating a positioning aid of the measuring device relative to a patient's bladder neck and verumontanum according to an embodiment.

FIG. 5 a schematic view of a distal end of a cystoscope having a light source and a measuring device configured to guide light from the light source according to an embodiment.

FIGS. 6a-c are schematic views that demonstrate locating an expander of the measuring device relative to a patient's bladder neck and verumontanum according to an embodiment.

FIGS. 7a-c are schematic views of markings provided on a barrel of the measuring device according to an embodiment.

FIGS. 8a-c are schematic views that demonstrate locating an expander of the measuring device relative to a patient's bladder neck and verumontanum using markings on a distal end of the measuring device according to an embodiment.

FIG. 9a is a schematic view of a measuring device having a ruler portion and a stabilizer according to an embodiment.

FIG. 9b is a schematic view of the measuring device of FIG. 9a that also features a supporter according to an embodiment.

FIGS. 10a-b are schematic views of alternative stabilizers according to embodiments.

FIGS. 11a-b are schematic views of a measuring device that may be used with a marker clip according to an embodiment.

FIGS. 11c-d are schematic views of a measuring device that features a ruler portion hinged to a marker clip according to an embodiment.

FIGS. 12a-b are schematic views that demonstrate determining prostatic urethral length using a measuring device having a ruler portion and a stabilizer according to an embodiment

### DETAILED DESCRIPTION

At the outset, it is to be understood that this disclosure is not limited to particularly exemplified materials, architectures, routines, methods or structures as such may vary. Thus, although a number of such options, similar or equivalent to those described herein, can be used in the practice or embodiments of this disclosure, the preferred materials and methods are described herein.

It is also to be understood that the terminology used herein is for the purpose of describing particular embodiments of this disclosure only and is not intended to be limiting.

The detailed description set forth below in connection with the appended drawings is intended as a description of exemplary embodiments of the present disclosure and is not intended to represent the only exemplary embodiments in which the present disclosure can be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the specification. It will be apparent to those skilled in the art that the exemplary embodiments of the specification may be practiced without these specific details. In some instances, well known structures and devices are shown in block diagram form in order to avoid obscuring the novelty of the exemplary embodiments presented herein.

For purposes of convenience and clarity only, directional terms, such as top, bottom, left, right, up, down, over, above, below, beneath, rear, back, and front, may be used with respect to the accompanying drawings. These and similar directional terms should not be construed to limit the scope of the disclosure in any manner.

Unless defined otherwise, all technical and scientific terms used herein have the same meaning as commonly understood by one having ordinary skill in the art to which the disclosure pertains. Moreover, as used in this specification and the appended claims, the singular forms “a,” “an” and “the” include plural referents unless the content clearly dictates otherwise.

Definitions: The terms “therapeutically effective displacement” or “therapeutically effective retraction” or “therapeutically effective expansion”, are used interchangeably herein and refer to an amount of displacement of prostatic tissue proximate to a restricted area of a urethra sufficient to increase the urethral lumen and treat, ameliorate, or prevent the symptoms of benign prostatic hyperplasia (BPH) or comorbid diseases or conditions, including lower urinary tract symptoms (LUTS), bladder outlet obstruction (BOO), benign prostatic obstruction (BPO), wherein the displacement of prostatic tissues exhibits a detectable therapeutic, prophylactic, or inhibitory effect. The effect can be detected by, for example, an improvement in clinical condition, or reduction in symptoms or absence of co-morbidities. Examples of clinical measures include a decrease in the international prostate symptom score (IPSS), reduction in post-void residual (PVR) volume of urine in the bladder after relief or increase in the maximum urinary flow rate (Qmax) or improvement in quality of life (QoL), improvement in sexual health (sexual health inventory for men or SHIM score, men's sexual health questionnaire or MSHQ score) after treatment. The precise distance or volume of the displacement of prostatic tissue will depend upon the subject's body weight, size, and health; the nature and extent of the enlarged or diseased prostatic condition and the size of the implant selected for placement in the patient.

As used herein, a patient “in need of treatment for BPH” is a patient who would benefit from a reduction in the presence of or resulting symptoms of enlarged prostatic tissue caused by a non-malignant enlarging of the prostate gland and related disorders, including LUTS, urinary outflow obstruction symptoms and luminal narrowing of the prostatic urethra. As used herein, the terms “implant” or “expander” or “device” refer to the prosthetic device that is implanted within the prostatic urethra to relieve LUTS associated or caused by BPH.

With respect to orientation of the various structures and anatomical references described herein, the term “proximal” and “distal” are relative to the perspective of the medical professional, such as a urologist, who is manipulating the delivery system of the disclosure to deploy the implants described herein. Accordingly, those features of the delivery system held by the hand of the urologist are at the “proximal” end and the assembled system and the implant, initially in its compressed configuration, is located at the “distal” end of the delivery system.

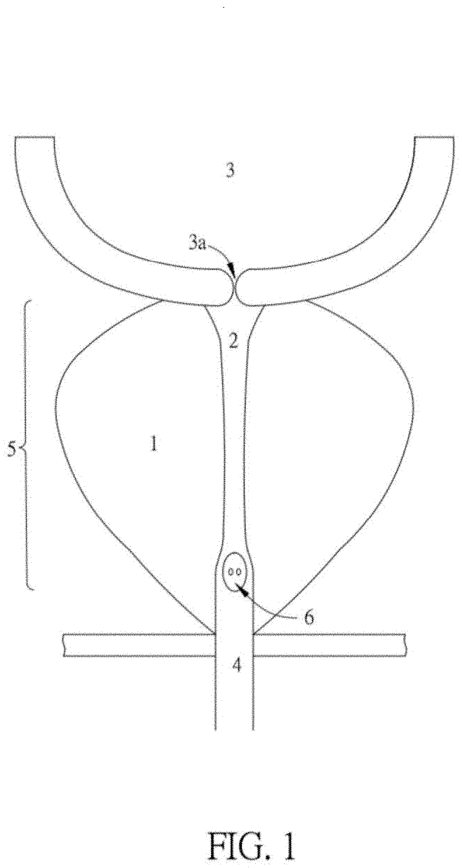

Referring to FIG. 1, a cross-section of the male anatomy shows the prostate gland 1 surrounding the prostatic urethra 2. The prostatic urethra 2, under normal conditions, provides fluid communication from urine stored in the bladder 3 to be expelled from the body under involuntary muscular control of the internal sphincter (or bladder neck) 3a and voluntary muscular control of the external sphincter 4. Normal or “true” prostate tissue of gland 1 surrounds the prostatic urethra 2 and, in the absence of disease, does not impinge on the patency of prostatic urethra 2. As noted above, it is desirable to determine prostatic urethral length (PUL) 5, which for the purposes of this disclosure may be defined as the distance between bladder neck 3a and verumontanum 6.

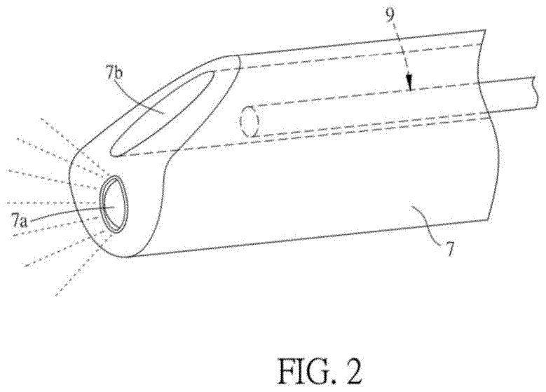

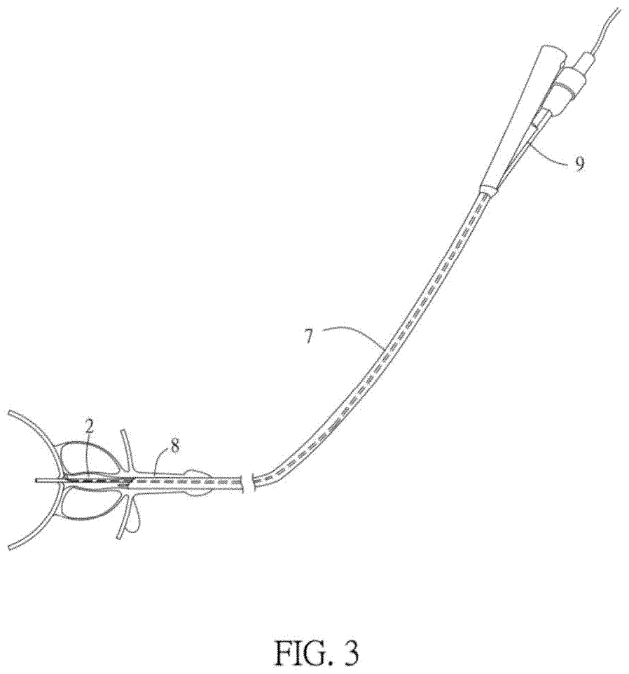

According to an embodiment of this disclosure, a medical device and method of measuring PUL 5 is disclosed. For example, in reference to FIGS. 2 and 3, a cystoscope 7 is generally used to examine bladder 3 or prostatic urethra 2 of patient for diagnosis, investigation or treatment in patients suffering from LUTS. Cystoscope 7 is equipped with a light source 7a, such as an LED, at its distal end to aid visualization. In use, the physician will insert cystoscope 7 into the penis 8 and through prostatic urethra 2. A measuring device 9 embodying aspects of this disclosure may be inserted into a lumen 7b of cystoscope 7 to determine the PUL 5, for example, before the implant deployment procedure. The measuring device 9 includes a marked stylet for providing intraluminal measurement of the specific anatomic length between two anatomic landmarks under visualization of scope (i.e., endoscope or cystoscope) by indicating relative translation movement of measuring device 9 within cystoscope 7. Accordingly, the physician can accurately measure PUL 5 and provide the information for selecting an appropriately sized interventional device. Similarly, the physician may also measure PUL 5 in conjunction with evaluating the need for surgical treatment, predicting expected symptoms of the patient and/or any other suitable investigative or diagnostic procedure.

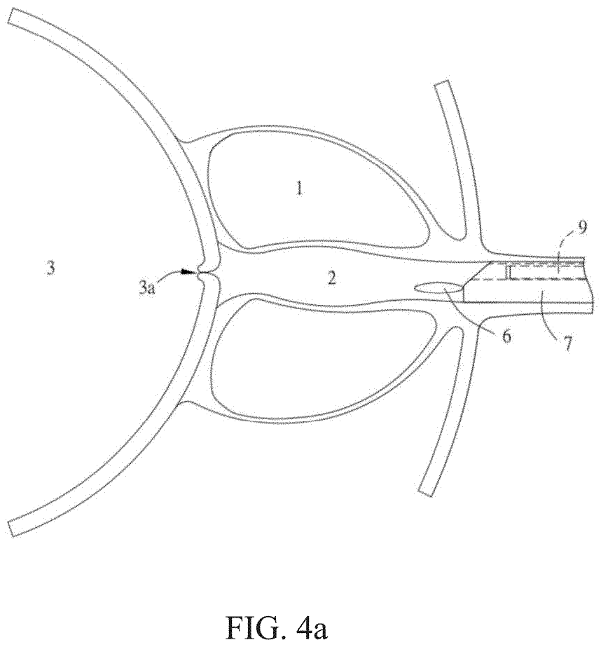

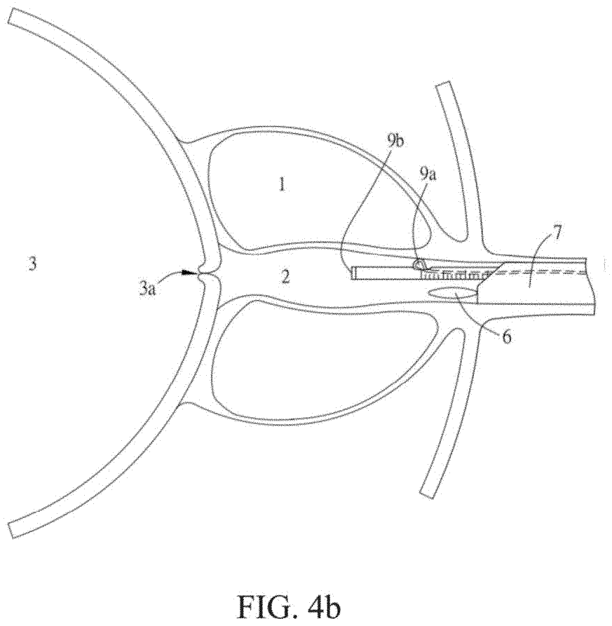

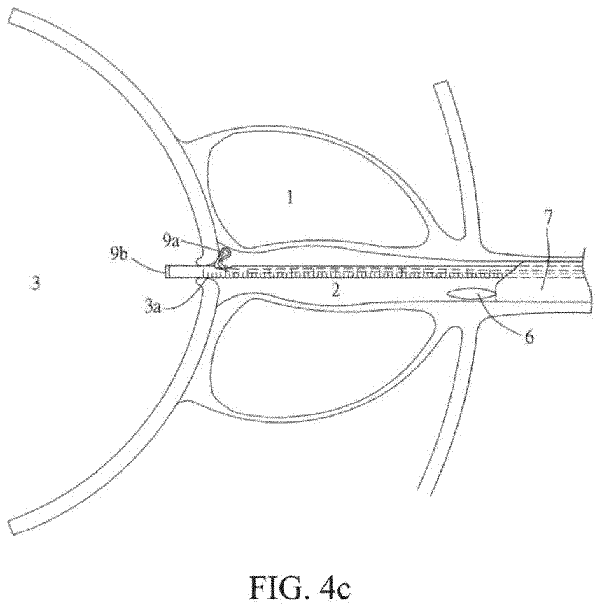

In use, measuring device 9 is initially positioned within a working channel 7b of cystoscope 7 so that the distal end of measuring device 9 is adjacent the distal end of cystoscope 7. The physician may then insert cystoscope 7 with measuring device 9 into urethra 2 until verumontanum 6 is seen through cystoscope 7 so that the distal end of cystoscope 7 (and the distal end of measuring device 9) is positioned at verumontanum 6. Next, the physician extends measuring device 9 from cystoscope 7 towards until the distal end of measuring device 9 is positioned at bladder neck 3a. A determination of the distance traveled may be obtained via the predetermined markings on measuring device 9, as schematically depicted in FIGS. 4a, 4b and 4c.

In the present embodiment, measuring device 9 is configured as a stylet having an elongated and tubular body, with a plurality of markings spaced apart on the outside surface denoting a suitable measurement of distance. The markings can be disposed on distal portion or proximal portion of stylet. The markings may represent any suitable unit of absolute length measurement. Alternatively, the markings can also indicate relative sizing of an implant (with small, medium, large marks, for example, or any other level of granularity) dependent on the intended use of measuring device 9.

To create a visible indicator from the physician's view, measuring device 9 of present embodiment may also include at least one positioning aid 9a disposed at or near the distal end of the stylet. In one embodiment, positioning aid 9a may be implemented as a visual cue, such as implemented as a protrusion from a side hole of the tubular body or any other projection. As will be appreciated, when PUL 5 is relatively longer, the physician may benefit from the enhanced visual indication of positioning aid 9a which functions as a “visual locator,” thereby facilitating positioning with respect to bladder neck 3a under view of cystoscope 7. The enhanced visibility of positioning aid 9a of the present embodiment with respect to bladder neck 3a helps the physician accurately position marking device 9 at the location of bladder neck 3a by confirming that positioning aid 9a is in the intended alignment.

In the present embodiment, the positioning aid 9a can be provided with high-contrast features for improved visual identification, such as by using different colors, different and/or enlarged visual indicators, lights, or any combination of these and other effects. Thus, when positioning aid 9a is present in the center of view, positioning aid 9a improves visibility to facilitate accurate positioning of marking device 9 relative to bladder neck 3a under visualization of cystoscope 7.

Alternatively, or in addition, positioning aid 9a may comprise a tactile cue for the physician to detect the location of bladder neck 3a. For example, such tactile feedback can be created by an expandable or deployable feature disposed at the distal end of the stylet of measuring device 9 in one embodiment. Prior to expansion, delivery of measuring device through cystoscope 7 is not impeded and then when expanded, the enlarged profile can engage with and thereby locate a proximal surface of bladder neck 3a. Accordingly, positioning aid 9a can be made of soft or semi-rigid material, such as suture material. In addition, it can transition from a low profile (or compressed) to a high profile (deployed or expanded) configuration to first facilitate delivery through working channel 7b of cystoscope 7 and then to improve engagement with bladder neck 3a to position the stylet. For example, the positioning aid 9a can be accommodated in the stylet and deployed from a side hole of measuring device 9 to function as a visual and/or tactile cue when deployed (as shown in FIG. 4b).

In an embodiment, measuring device 9 with positioning aid 9a is passed through the prostatic urethra 2 towards bladder 3 by being advanced from working channel 7b of cystoscope 7. When positioning aid 9a is traveling within prostatic urethra 2, positioning aid 9a may be restricted by the urethral lumen, maintaining it in a first compressed or low profile configuration. Accordingly, when the end of measuring device 9 emerges from prostatic urethra 2 and is adjacent bladder neck 3a, positioning aid 9a may then be automatically released from its restricted configuration into the relatively high profile or expanded configuration when measuring device 9 is not constrained by the relatively narrow passage of the urethral lumen. As will be appreciated, the transition of positioning aid 9a assists the physician when determining the location of the end of measuring device 9.

Alternatively, as shown in FIG. 4c, measuring device 9 can further include an emitter 9b for light conveyed through the tubular body of the stylet by an optical fiber or other suitable component. Emitter 9b may be disposed at the distal end of the stylet of measuring device 9, adjacent positioning aid 9a, to provide illumination and enhance visualization of positioning aid 9a by guiding light from a proximal end of measuring device 9. For example, measuring device 9 may provide a blinking light via emitter 9b at the distal end of the tubular body to supply an additional source of light and facilitate positioning of positioning aid 9a with respect to bladder neck 3a. Further, light source 9b at the distal end of measuring device 9 can also illuminate the interior space of urethra 2 and assist the physician when measuring PUL 5.

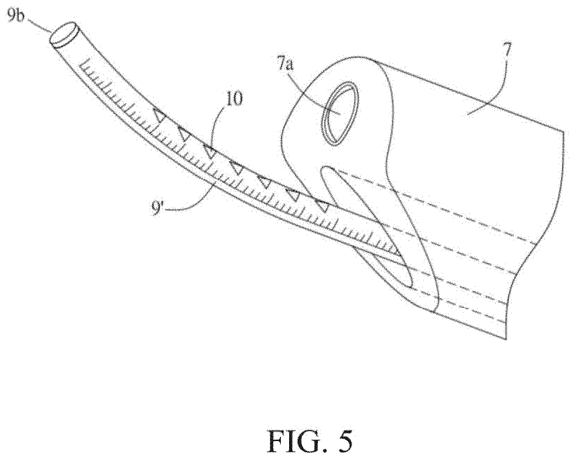

In yet another embodiment as schematically depicted in FIG. 5, an alternative measuring device 9′ can be constructed from an optical fiber with the measurement indications on its surface. The optical fiber is utilized to effectively guide light from light source 7a of cystoscope 7 distally to emitter 9b. In addition, the optical fiber may have a plurality of notches 10 formed on the elongated body of the optical fiber. The notches 10 have a structure configured to direct or guide the light emitted from the light source 7a of cystoscope 7 to the core of optical fiber. Therefore, measuring device 9′ with notches 10 of the embodiment can more fully utilize light source 7a to convey light to its distal end when measuring device 9′ is advanced beyond the distal end of cystoscope 7 when positioned at verumontanum 6.

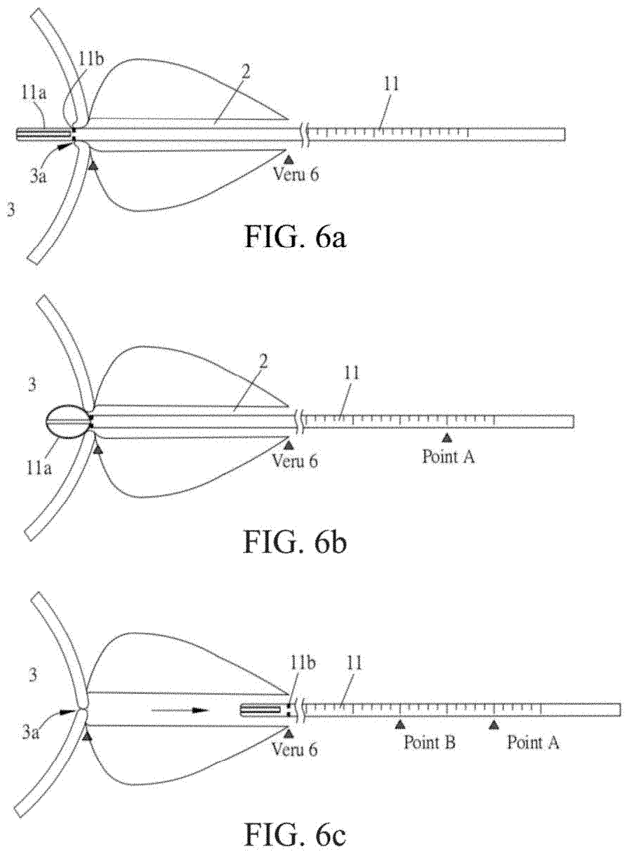

In some scenarios, visualization may be limited due to intraluminal curvature or enlarged tissue (i.e., enlarge prostate in urological application) distal the end of cystoscope 7. Accordingly, the techniques of this disclosure include a measuring device 11 having an expander 11a disposed at the distal end of the elongated body as schematically depicted in FIGS. 6a-6c to function as a positioning aid. Expander 11a is configured to be reversibly expandable after the end of measuring device 11 is disposed within bladder 3. As shown in FIG. 6a, expander 11a is first introduced into bladder 3 in its unexpanded configuration and is then expanded as indicated by FIG. 6b, thereby providing an anchor force caused by resistance at bladder neck 3a when the physician attempts to withdraw measuring device 11 after deploying expander 11a. This anchor force provides a tactile cue, allowing the physician to confirm the position of bladder neck 3a. Expander 11a is then collapsed to allow measuring device 11 to be retrieved by the physician. In this embodiment, measuring device 11 further has an indicator 11b positioned at distal end of measuring device 11, adjacent to the proximal end of expander 11a so that it corresponds to the position of bladder neck 3a when a withdrawal force is exerted on measuring device 11 and deployed expander 11a resists entry into prostatic urethra 2. Measuring device 11 also features measurement markings discussed above for determining PUL 5 on the proximal portion of measuring device 11 for reading by the physician outside the patient's body. Once the physician locates bladder neck 3a, such as by resistance provided by expander 11a, the expander is collapsed and measuring device 11 is withdrawn until indicator 11b is located at verumontanum 6, which serves as a landmark for measuring PUL 5 the length. Alternatively, expander 11a can be formed by wire, balloon, strip or other suitable structures having the capability to transition from expanded and collapsed configurations.

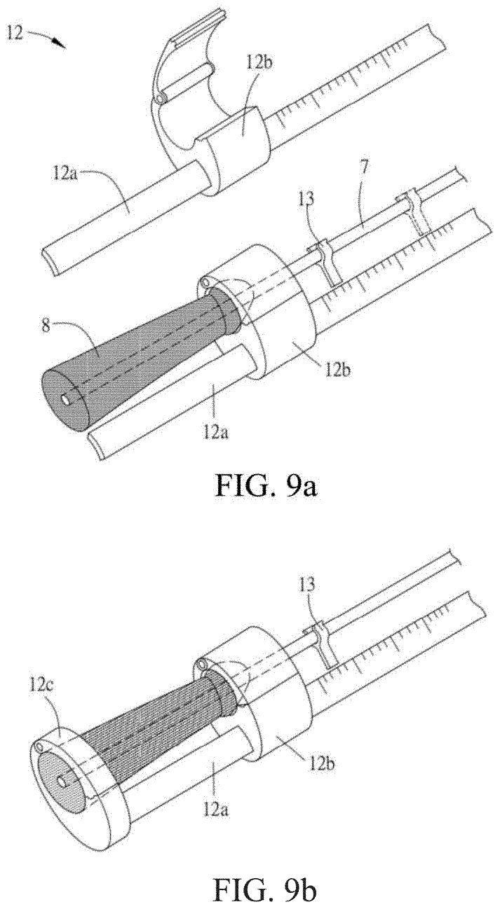

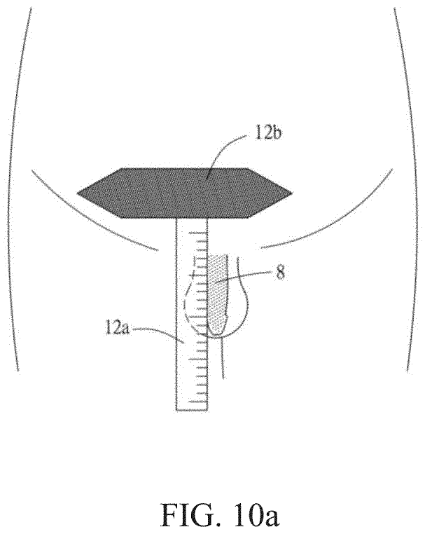

As schematically indicated in the figures, the measurement may be taken as a differential in the proximal markings between the two noted locations. Exemplary usage of measuring device 11 may include deployment within working channel 7b of cystoscope 7, with expander 11b in its collapsed configuration. The physician then inserts cystoscope 7 with measuring device 11 into prostatic urethra 2 until verumontanum 6 is seen through cystoscope 7. With the distal end of cystoscope 7 positioned at verumontanum 6, the physician extends marked measuring device 11 from cystoscope 7 into the bladder 3 (as shown in FIG. 6a) and expands the expander 11a into its second configuration, i.e., expanded configuration (as shown in FIG. 6b). The physician then exerts a withdrawal force to measuring device 11 to confirm the location of bladder neck 3a with tactile feedback. Concurrently, the physician notes the measurement marking with respect to a stationary reference, such as tip of penis 8, to indicate bladder neck 3a location (schematically indicated as Point A in FIG. 6b). The physician then collapses expander 11a and withdraws measuring device 11 until indicator 11b is aligned with verumontanum 6 under cystoscope view. Noting the measurement marking using the same stationary reference (schematically indicated as Point B shown in FIG. 6c) enables determination of PUL 5 as the distance between Point A and Point B represents the distance between bladder neck 3a and verumontanum 6. In this embodiment, PUL 5 can be easily determined via the portion of measuring device 11 exposed outside of body. In alternative embodiments, the external measurement can refer to FIGS. 9b, 11b, and 12b as discussed below.

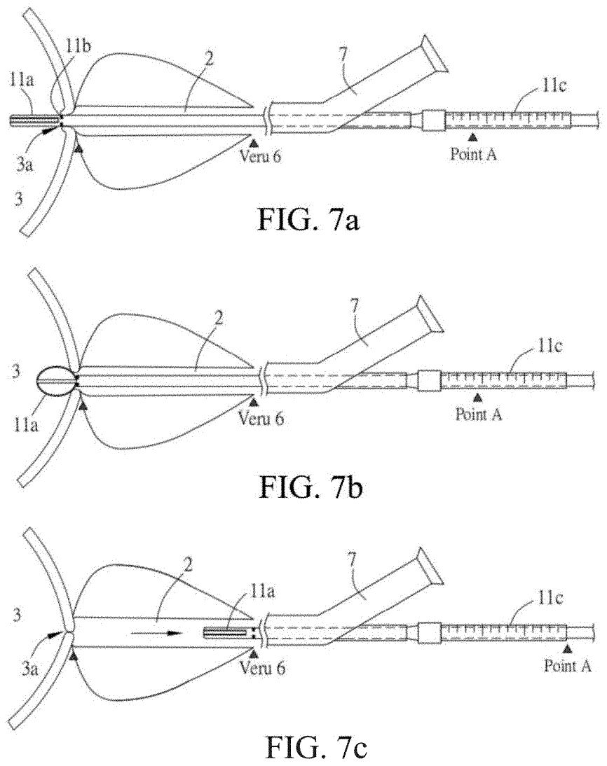

A variation of measuring device 11 is disclosed in reference to FIGS. 7a-7c. In this embodiment, measuring device 11 again comprises an elongated body with an expander 11a disposed at the distal end of elongated body and indicator 11b proximally adjacent. Additionally, the elongated body is disposed within a barrel 11c. As noted above, expander 11a is an expandable element which can serve as an anchor and provide the user tactile feedback to locate bladder neck 3a. As shown, the measuring markings are located on barrel 11c, which may be made of optically transparent material so that movement of the elongated body can be seen through barrel 11c. In use, the physician transitions expander 11a to its expanded configuration after measuring device 11 is advanced into bladder 3 with aid of cystoscope 7. The physician notes a first location of Point A on the elongated body via markings on the barrel 11c, as shown in FIG. 7b. Then, the physician can collapse and withdraw the measuring device 11 until indicator 11b aligns with the verumontanum 6 similar to the embodiments described above, with Point A at a new position on the elongated body. Thus, the physician can determine the length between the bladder neck and the verumontanum via the movement distance of a mark, e.g., Point A, on the measuring device 11. In the present embodiment, barrel 11c can be connected or otherwise secured to an entry of working channel of cystoscope 7. In this case, barrel 11c is stationary and the elongated body of measuring device 11 can be moved in relation to the barrel 11c.

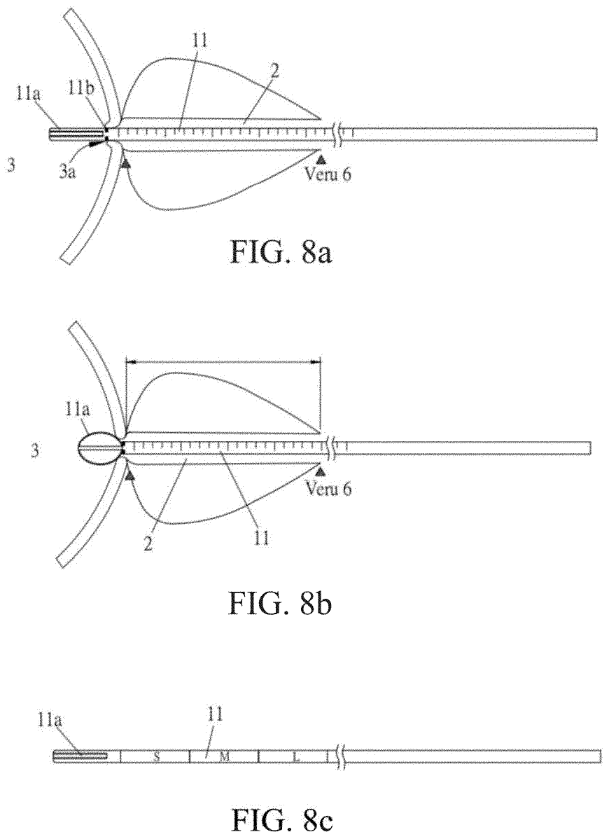

Accordingly, depending on the embodiment, the physician can read the markings not only from the outside of patient body, as shown in FIGS. 6 and 7, but also in the lumen of urethra via the view of cystoscope 7. Therefore, referring to FIG. 8, the measuring device 11 has markings on the distal portion of elongated body. When expander 11a is expanded and used to locate bladder neck 3a, the physician can read the markings directly using verumontanum 6 as landmark. In the present embodiment, the scale of markings can be in different forms. Further, as noted above, the markings may indicate the relative size of a device to be implanted rather than absolute measurements as exemplified by FIG. 8.

Since urological clinical practice may employ a flexible or rigid cystoscope to access a patient's urethra for investigation or examination, the techniques of this disclosure also include a medical device and method for measuring prostatic urethral length externally. Principally, a ruler with stabilizer and at least one marker is provided for the physician to identify the movement of the cystoscope for PUL 5 measurement outside the urethra.

For example, a measuring device 12 may include a ruler 12a having markings, a stabilizer 12b configured to position the ruler 12a with respect to a patient and at least one marker clip 13 configured to mark the different locations of cystoscope 7 inserted into penis 8 as shown in FIGS. 9a and 9b. Prior to insertion of cystoscope 7, stabilizer 12b is used externally to engage with the glans or penis to stabilize and otherwise minimize movement of the penis during investigation or examination. In another embodiment, the measuring device 12 can further include a supporter 12c to engage a more distal portion of penis 8, as shown in FIG. 9b. Thus, compression or stretching of the urethra during investigation or examination can be minimized by way of providing additional stabilization via stabilizer 12b and optionally supporter 12c to help ensure accuracy of measurement.

During an illustrative procedure to measure PUL 5, the physician first inserts cystoscope 7 to locate bladder neck 3a. Once the position of bladder neck 3a is confirmed, the physician notes the position of marker clip 13 on the shaft of cystoscope 7 relative to the markings of ruler 12a (analogous to Point A in the embodiments discussed above). Marker clip 13 is positioned outside and next to urethra orifice as the reference point. Then, the physician pulls back cystoscope 7 until verumontanum 6 is seen and the new position of marker clip 13 (shown in phantom in FIG. 9a) relative to ruler 12a. Accordingly, the distance between the two positions of marker clip 13 will indicated PUL 5, the distance between bladder neck 3a and verumontanum 6. In the present embodiment, the marker clip can be made of elastic material to prevent the damage of cystoscope 7. Alternatively, a predefined indicator may be provided on cystoscope 7.

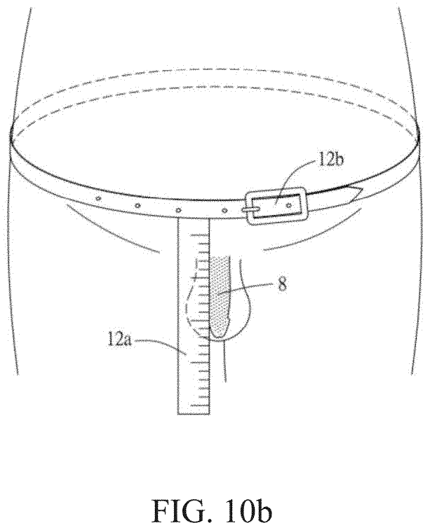

Alternatively, as schematically indicated in FIG. 10a, stabilizer 12b of measuring device 12 can configured as a sticker or patch to be adhered to the hypogastrium skin. Thus, ruler 12a can be help in a straight position for measuring and stabilizer 12b will not stretch penis 8 or urethra 2 during measurement. As an alternative embodiment, stabilizer 12b can be in form of belt, as shown in FIG. 10b.

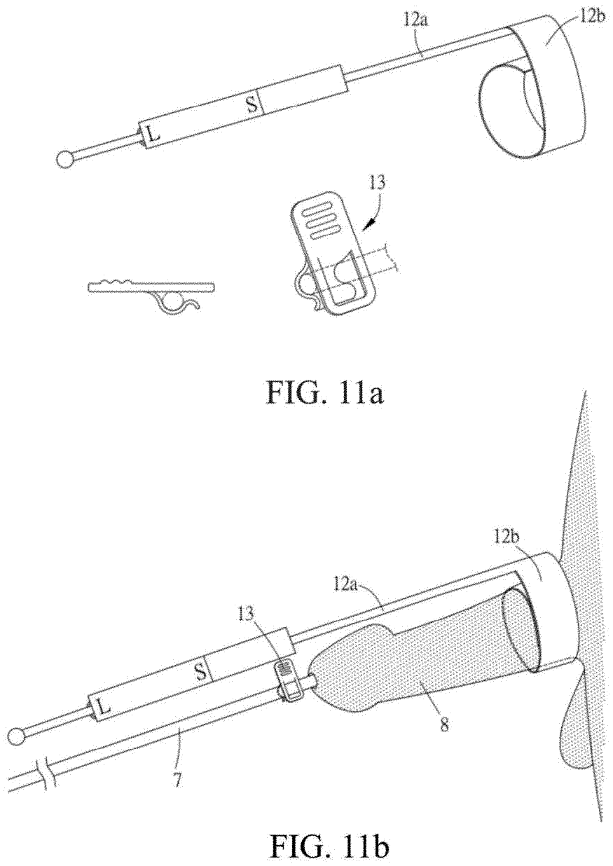

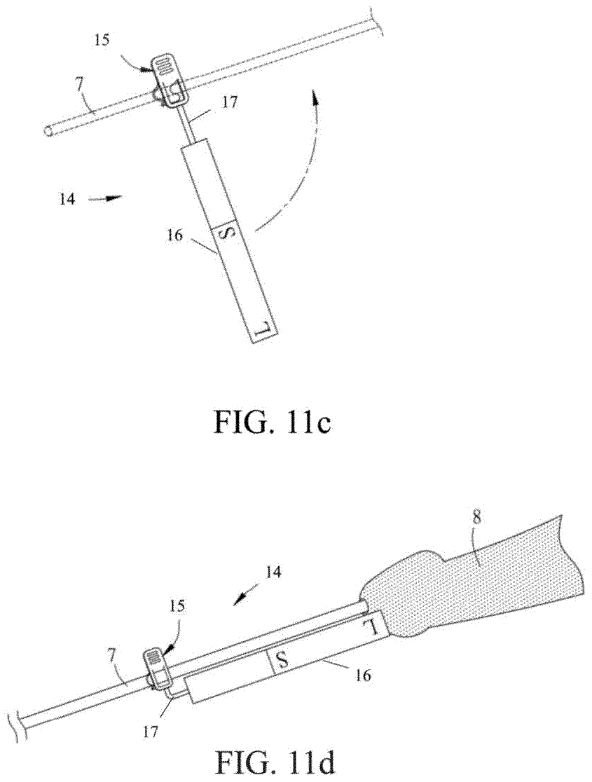

Referring to FIGS. 11a-11b, stabilizer 12b of measuring device 12 can be a sticker and adhere to the bottom of penis to minimize stretching of penis 8 or urethra 2 during measurement. As mentioned above, marker clip 13 can be employed to track the location of cystoscope 7 when its distal end is positioned at bladder neck 3a and at verumontanum 6. As noted above, the scale of markings on ruler 12a can be in different forms to achieve different objectives, such as by indicating the relative size of a device to be implanted or by representing absolute length.

In yet another embodiment, a measuring device 14 comprises a clip 15 that may be removably attached to a desired location on cystoscope 7 and a ruler 16 connected to the marker clip by hinge 17, as shown in FIGS. 11c and 11d. Since the measuring device 12 is utilized outside of body and not involved interaction with patient, the urethra compression/stretching will be prevented. In addition, the accuracy of measurement can be assured. In use, PUL 5 may be measured using measuring device 14 by first inserting cystoscope 7 to locate bladder neck 3a. Once the position of bladder neck 3a is confirmed, the physician secures marker clip 15 to the shaft of cystoscope 7 at a desired reference position, such as just outside and next to the urethra orifice. The physician then withdraws cystoscope 7 until verumontanum 6 is seen. By pivoting ruler 16 on hinge 17 until it is aligned with the shaft of cystoscope 7, the markings on ruler 16 can be compared to the same reference position to determine PUL 5.

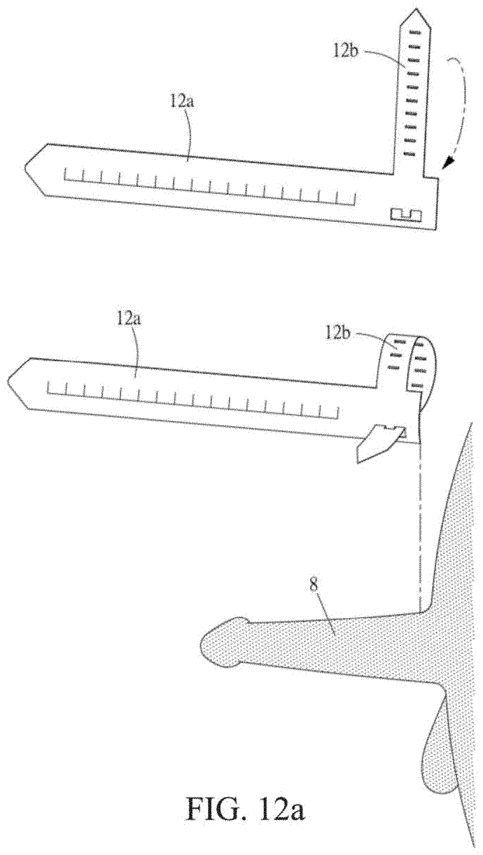

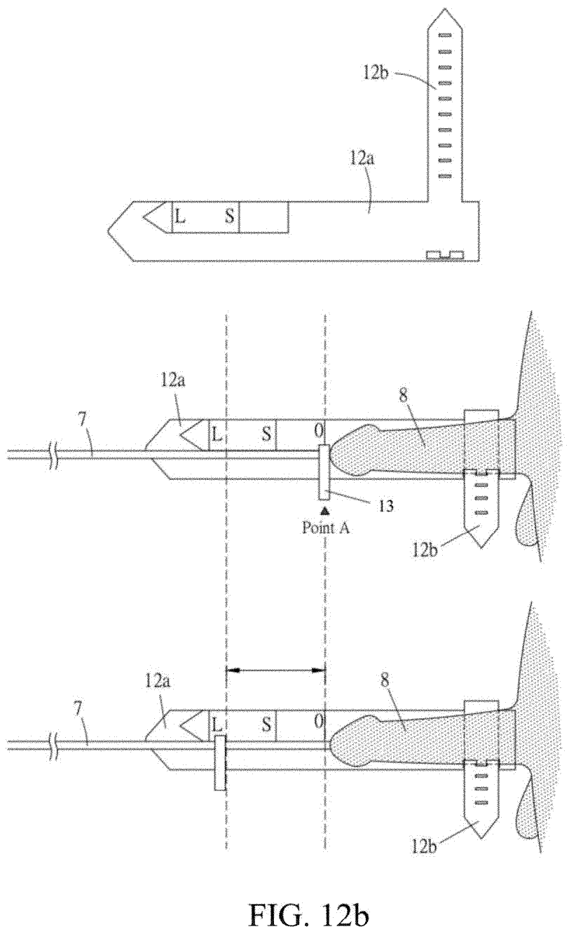

Still further, stabilizer 12b of measuring device 12 can be in a form of strip which is able to be adjusted in tightness to fit patient's penis 8 according to the embodiment shown in FIGS. 12a and 12b. In this case, stabilizer 12b is strapped to the base of penis 8. In use, marker clip 13 or a preexisting indicator on the shaft of cystoscope 7 may be used as discussed above to take the appropriate measurements from ruler 12a to measure PUL 5.

The exemplary embodiments disclosed above are merely intended to illustrate the various utilities of this disclosure. It is understood that numerous modifications, variations and combinations of functional elements and features of the present disclosure are possible in light of the above teachings and, therefore, within the scope of the appended claims, the present disclosure may be practiced otherwise than as particularly disclosed and the principles of this disclosure can be extended easily with appropriate modifications to other applications.

All patents and publications are herein incorporated for reference to the same extent as if each individual publication was specifically and individually indicated to be incorporated by reference. It should be understood that although the present disclosure has been specifically disclosed by preferred embodiments and optional features, modification and variation of the concepts herein disclosed may be resorted by those skilled in the art, and that such modifications and variations are considered to be within the scope of this disclosure.

What is claimed is:

1. A measuring device for determining prostatic urethral length, comprising:
a ruler portion having markings configured to indicate relative translational movement of a cystoscope; and
a clip connected to the ruler portion configured to be removably attached to the cystoscope.

2. The measuring device of claim 1, wherein the ruler portion is connected to the clip by a hinge.

3. The measuring device of claim 2, wherein the hinge is configured to allow the ruler portion to align longitudinally with the cystoscope when the clip is attached.

4. The measuring device of claim 1, wherein markings of the ruler portion represent absolute measurements.

5. The measuring device of claim 1, wherein markings of the ruler portion represent relative measurements.

6. The measuring device of claim 1, wherein the clip is configured to be attached to the cystoscope at a reference position relative to a patient.

7. A method for determining prostatic urethral length, comprising:
locating a distal end of the cystoscope at a patient's bladder neck such that the cystoscope is at a first translational position;
releasably attaching a ruler portion of a measuring device having markings configured to indicate relative translational movement to the cystoscope using a clip;
withdrawing the cystoscope until the distal end is located at the patient's verumontanum such that the cystoscope is at a second translational position;
corresponding the second position of the cystoscope to the markings to determine prostatic urethral length.

8. The method of claim 7, wherein the ruler portion is releasably attached to a reference position relative to the patient when the distal end of the cystoscope is located at the patient's bladder neck.

9. The method of claim 8, wherein corresponding the second position of the cystoscope to the markings to determine prostatic urethral length comprises comparing the markings of the ruler portion with the reference position relative to the patient.

10. The method of claim 7, further comprising pivoting the ruler portion at a hinge of the clip to align the ruler portion longitudinally with the cystoscope when corresponding the second position of the cystoscope to the markings.
